(19) United States
(12) Patent Application Publication
(10) Pub. No.: US 2011/0183912 A1
(43) Pub. Date: Jul. 28, 2011

(54) AGENT FOR PROMOTING NEURONAL DIFFERENTIATION AND METHOD THEREFOR

(75) Inventors: Hideyuki Okano, Tokyo (JP); Takuya Shimazaki, Tokyo (JP); Hayato Naka, Tokyo (JP)

(73) Assignee: Keio University, Minato-ku (JP)

(21) Appl. No.: 13/058,621

(22) PCT Filed: Jul. 8, 2009
(86) PCT No.: PCT/JP2009/003195

§ 371 Date: Apr. 8, 2011

Related U.S. Application Data
(60) Provisional application No. 61/088,521, filed on Aug. 13, 2008.

(51) Int. Cl. A61K 38/02; C12N 5/0797; A61K 31/7088; A61P 25/28; A61P 25/00

(52) U.S. Cl. 514/17.8; 435/377; 514/44 A; 514/17.7

(57) ABSTRACT

An agent for promoting neuronal differentiation of a neural stem/progenitor cell includes an inhibitor of function of a COUP-TFI protein and/or a COUP-TFII protein. To promote neuronal differentiation of a neural stem/progenitor cell, the agent is administered to the neural stem/progenitor cell to inhibit function of a COUP-TFI protein and/or a COUP-TFII protein.

### CROSS REFERENCE TO RELATED APPLICATIONS

This application claims the benefit of priority to U.S. provisional application 61/088,521, filed on Aug. 13, 2008, which is incorporated herein by reference.

### TECHNICAL FIELD

The present invention relates to agents for promoting neuronal differentiation of neural stem/progenitor cells, and methods therefor.

### BACKGROUND ART

In order to utilize stem cells having multipotency in the field of regenerative medicine, it is necessary to differentiate the stem cells into a certain cell type. Neural stem/progenitor cells (NCPCs) can differentiate from embryonic stem cells (ES cells) in vitro (Japanese Patent Application Laid-open Publication No. 2002-291469), and also can be isolated from an embryonic brain (Reynolds, B. A. and Weiss, S. Science vol. 255, pp. 1707-1710 (1992)). The NCPCs are capable of differentiating into both neurons and glial cells, and methods for inducing the differentiation into either neurons or glia have been developed.

For example, by administering an IL-6 family cytokine (Koblar, S. A., et al. Proc. Natl. Acad. Sci. U.S.A. vol. 95, pp. 3178-3181 (1998)) or BMP2/4 (Gross, R. E., et al. Neuron vol. 17, pp. 595-606 (1996)) to NCPCs, they can be induced to differentiate into glial cells.

However, mechanisms of the differentiation into neurons, especially of sequential differentiation into different types of neurons have been scarcely elucidated, and thus the techniques for differentiating specifically into neurons have not been as much developed as those for glial cells.

### SUMMARY OF INVENTION

### Technical Problem

The present invention is intended to provide agents for promoting neuronal differentiation of NSPCs, and methods therefor.

### Solution to Problem

In one embodiment of the present invention, an agent for promoting neuronal differentiation of an NSPC includes an inhibitor of function of a COUP-TFI protein and/or a COUP-TFII protein. The inhibitor may inhibit expression(s) of (a) gene(s) encoding a COUP-TFI protein and/or a COUP-TFII protein. The inhibitor may be a nucleic acid capable of inhibiting expression of the gene. The nucleic acid may be an shRNA or an siRNA. Further, the inhibitor may be a competitive inhibition mutant of a COUP-TFI protein or a COUP-TFII protein, and the competitive inhibition mutant may include a transcription activation domain and a DNA-binding domain of a COUP-TFI protein or a COUP-TFII protein. The NSPC may be derived from an embryonic stem cell or an induced pluripotent stem cell, and the NSPC may form a primary neurosphere. The neuron that differentiates by any agent described above may be an Isl-1-positive neuron, a DARPP-32-positive neuron or a Tbr1-positive neuron. Furthermore, the differentiated neuron may be a cholinergic neuron, a GABAergic neuron and a glutamatergic neuron.

In another embodiment of the present invention, a method for promoting neuronal differentiation of an NSPC includes inhibiting function of a COUP-TFI protein and/or a COUP-TFII protein in the NSPC. The method may include inhibiting expression(s) of (a) gene(s) encoding a COUP-TFI protein and/or a COUP-TFII protein in the NSPC. In this method, a nucleic acid molecule capable of inhibiting expression of the gene may be introduced into the NSPC, and the nucleic acid molecule may be an shRNA or an siRNA. Further, a competitive inhibition mutant of a COUP-TFI protein or a COUP-TFII protein may be introduced into the NSPC. The competitive inhibition mutant may include a transcription activation domain and a DNA-binding domain of a COUP-TFI protein or a COUP-TFII protein. The NSPC may be derived from an embryonic stem cell or an induced pluripotent stem cell, and the NSPC may form a primary neurosphere. The neuron that differentiates by any method described above may be an Isl-1-positive neuron, a DARPP-32-positive neuron and a Tbr 1-positive neuron. Furthermore, the differentiated neuron may be a cholinergic neuron, a GABAergic neuron and a glutamatergic neuron.

In another embodiment of the present invention, a pharmaceutical composition includes an inhibitor of function of a COUP-TFI protein and/or a COUP-TFII protein. The inhibitor may inhibit expression(s) of (a) gene(s) encoding a COUP-TFI protein and/or a COUP-TFII protein. The inhibitor may be a nucleic acid capable of inhibiting expression of the gene. The nucleic acid may be an shRNA or an siRNA. Further, the inhibitor may be a competitive inhibition mutant of a COUP-TFI protein or a COUP-TFII protein, and the competitive inhibition mutant may include a transcription activation domain and a DNA-binding domain of a COUP-TFI protein or a COUP-TFII protein. The inhibitor may inhibit the function of a COUP-TFI protein and/or a COUP-TFII protein in an NSPC. The NSPC may be derived from an embryonic stem cell or an induced pluripotent stem cell, and the NSPC may form a primary neurosphere. The neuron that differentiates by any pharmaceutical composition described above may be an Isl-1-positive neuron, a DARPP-32-positive neuron and a Tbr1-positive neuron. Furthermore, the differentiated neuron may be a cholinergic neuron, a GABAergic neuron and a glutamatergic neuron. This pharmaceutical composition may be administered to a patient with brain ischemia, traumatic brain injury, Huntington's disease and Alzheimer's disease.

In another embodiment of the present invention, a method for treating a neurological disorder includes inhibiting function of a COUP-TFI protein and/or a COUP-TFII protein in a NSPC. The method may include inhibiting expression(s) of (a) gene(s) encoding a COUP-TFI protein and/or a COUP-TFII protein in the NSPC. In this method, a nucleic acid molecule capable of inhibiting expression of the gene may be introduced into the NSPC, and the nucleic acid molecule may be an shRNA or an siRNA. Further, a competitive inhibition mutant of a COUP-TFI protein or a COUP-TFII protein may be introduced into the NSPC. The competitive inhibition mutant may include a transcription activation domain and a DNA-binding domain of a COUP-TFI protein or a COUP-TFII protein. The NSPC may be derived from an embryonic stem cell or an induced pluripotent stem cell, and the NSPC may form a primary neurosphere. The neuron that differentiates by any method described above may be an Isl-1-positive neuron, a DARPP-32-positive neuron and a Tbr1-positive neuron. Furthermore, the differentiated neuron may be a cholinergic neuron, a GABAergic neuron and a glutamatergic neuron. The neurological disorder may be brain ischemia, traumatic brain injury, Huntington's disease and Alzheimer's disease.

### BRIEF DESCRIPTION OF DRAWINGS

FIG. 1 is a graph showing the inhibition of the expression of the COUP-TF protein by knockdown of the Coup-tfI gene and/or Coup-tfII gene in an example according to the present invention.

FIG. 2 is photographs showing the morphology of NSPCs in which the Coup-tfI gene and/or the Coup-tfII gene has been knocked down in an example according to the present invention.

FIG. 3 is a graph showing the ratios of neurons, astrocytes and oligodendrocytes which appeared when differentiation of the NSPCs in which the Coup-tfI gene and/or the Coup-tfII gene had been knocked down was induced in an example according to the present invention.

FIG. 4 is a graph showing the ratio of Isl-1 positive neurons that appeared when the NSPCs in which the Coup-tfI gene and the Coup-tfII gene had been knocked down were passaged and their differentiation was induced in an example according to the present invention.

FIG. 5 is a graph showing the ratios of neurons, oligodendrocyte progenitor cells (OPCs) and other cells (Others) which differentiated from the brain cells, into which shRNA KDI+II had been introduced at E10.5, in the mouse brain at E16.5 in an example according to the present invention.

FIG. 6 is a graph showing the ratios of neurons, astrocytes and other cells (Others) which differentiated from the brain cells, into which shRNA KDI+II had been introduced at E12.5, in the mouse brain at P20 in an Example according to the present invention.

FIG. 7 is graphs showing the ratios of Isl-1 positive neurons, DARPP-32 positive neurons and Tbr1 positive neurons, which appeared when the Coup-tfI gene and the Coup-tfII gene had been knocked down in vivo in an example according to the present invention.

FIG. 8 is a schematic diagram showing the structures of the fusion proteins to be used in the experiment of the competitive inhibition of the DNA binding function of the COUP-TF protein in an example according to the present invention.

FIG. 9 is a graph showing the ratios of neurons that appeared when the NSPCs, in which the DNA binding function of the COUP-TF protein was competitively inhibited, were induced to differentiate in an example according to the present invention.

### DESCRIPTION OF EMBODIMENTS

Embodiments of the present invention accomplished based on the above-described findings are hereinafter described in detail by giving Examples. Where there is no particular explanations in embodiments or Examples, methods described in standard sets of protocols such as J. Sambrook, E. F. Fritsch & T. Maniatis (Ed.) Molecular cloning, a laboratory manual (3rd edition) Cold Spring Harbor Press, Cold Spring Harbor, N.Y. (2001); F. M. Ausubel, R. Brent, R. E. Kingston, D. D. Moore, J. G. Seidman, J. A. Smith, K. Struhl (Ed.) Current Protocols in Molecular Biology, John Wiley & Sons Ltd., or their modified/changed methods are used. When using a commercial reagent kit and/or a measuring apparatus, protocols attached to them are used unless otherwise explained.

The object, characteristics, advantages of the present invention as well as the idea thereof are apparent to those skilled in the art from the descriptions given herein, and the present invention can be easily conducted by those skilled in the art based on the descriptions given herein. It should be understood that the embodiments and specific examples of the invention described herein below are to be taken as preferred examples of the present invention. These descriptions are only for illustrative and explanatory purposes and are not intended to limit the present inventions to these embodiments or examples. It is further apparent to those skilled in the art that various changes and modifications may be made based on the descriptions given herein within the intent and scope of the present invention disclosed herein.

### <Neural Stem/Progenitor Cells (NSPCs)>

The agent according to the present invention is used for promoting neuronal differentiation of NSPCs and contains an inhibitor of function of COUP-TFI protein and/or COUP-TFII protein. In this specification, promotion of neuronal differentiation of NSPCs means a phenomenon in which the ratio of glia decreased and the ratio of neurons increased in differentiation of the NSPCs by administering the agent for promoting neuronal differentiation. The NSPCs may be either cells present in vivo or in vitro.

As the NSPCs are present in vivo in the cerebral cortex during embryonic/fetal stages as well as in the hippocampus and lateral ventricle during adult stage of human or non-human vertebrates, the agent for promoting neuronal differentiation may be administered to an individual animal to promote neuronal differentiation of the NSPCs in vivo.

The NSPCs in culture may be prepared by isolating and culturing the NSPCs in vivo. Alternatively, they may be prepared by differentiating embryonic stem cells (ES cells) or induced pluripotent stem cells (iPS cells) into the NSPCs. The method for inducing differentiation of ES cells is disclosed in Japanese Patent Application Laid-open Publication No. 2002-291469 in detail, which is herein incorporated by reference. This method can be applied to iPS cells as well.

To improve differentiation potential into neurons when inducing differentiation, it is preferable to allow ES cells or iPS cells to form embryoid bodies (EBs) in advance, differentiate them into the NSPCs and then administer the agent to the NSPCs under the culturing condition. The formation of the EBs can be achieved by, for example, culturing ES cells or iPS cells in the presence of retinoic acid or Noggin protein. The retinoic acid may be added at a low level (10⁻⁹M to 10⁻⁶ M) to the medium. In the case of Noggin protein, the Xenopus noggin gene may be introduced into cultured mammalian cells to express the Noggin protein transiently and then the culture supernatant may be recovered and used as it is (1 to 50% (v/v)), or alternatively, recombinant Noggin protein (final concentration of approx. 1 ug/ml) may be used.

By dissociating the EBs thus obtained and culturing them in suspension in a serum-free culture medium supplemented with FGF-2 (10 to 100 ng/ml) and sonic hedgehog protein (1 to 20 nM), the NSPCs can differentiate and grow in a form of neurospheres. The addition of sonic hedgehog protein can improve the efficiency of the induction of differentiation from the NSPCs into motoneuron precursor cells as well as the efficiency of growth, and indeed a subsequent culture allows the cells to differentiate into motoneurons as well as into GABAergic neurons. As for the serum-free medium, it is preferable to use DMEM medium supplemented with glucose, glutamine, insulin, transferrin, progesterone, putrescine, selenium chloride, heparin and the like in addition to the above-mentioned supplements, and more preferable to use DMEM:F12 medium. The cells are preferably cultured under the condition of 5% CO₂ at 35 to 40° C. for 7 to 9 days.

The neurospheres directly derived from EBs are referred to as primary neurospheres. The primary neurospheres can be dissociated and cultured under the same condition to form the neurospheres again, which are referred to as secondary neurospheres. Hereinafter neurospheres that underwent at least one passage under culturing condition are referred to as secondary neurospheres.

By culturing the primary neurospheres in a general differentiation medium, mainly cholinergic neurons including motoneurons and GAB Aergic neurons are induced to differentiate. As for the medium for inducing differentiation, it is preferable to use the DMEM:F12 medium supplemented with glucose, glutamine, insulin, transferrin, progesterone, putrescine and selenium chloride, which corresponds to the medium for growing neural stem cells from which FGF and heparin are omitted. Sonic hedgehog protein may or may not be added in the medium. It is preferable that the cells are cultured under the condition of 5% CO₂ at 35 to 40° C. for 5 to 7 days. If secondary neurospheres are cultured under the same condition, they differentiate not only into neurons that are mainly cholinergic neurons including motoneurons and GABAergic neurons but also into glial cells.

<Inhibitor of the Function of COUP-TF Protein>

COUP-TF protein (Coup-tfI and Coup-tfII are herein collectively referred to as Coup-tf in either case of a gene or a protein) may be derived from a vertebrate such as human and mouse. Although it has preferably the same origin as the NSPCs to be targeted, their origins may be different as long as the inhibitor can inhibit the function of the COUP-TF protein in the cell to be targeted.

The inhibitor may inhibit the function of the COUP-TF protein in any mechanism as long as the inhibition is achieved; it may reduce the activity of the COUP-TF protein directly, or it may inhibit the expression of the COUP-TF protein in a cell. Further, the inhibitor to be used is not particularly limited as long as it is a substance capable of inhibiting the function of the COUP-TF protein; it may be a low molecular compound, or a high molecular compound such as a nucleic acid or a protein.

Examples of the inhibitor nucleic acid include siRNAs, shRNAs and antisense RNAs capable of inhibiting the expression of the gene encoding the COUP-TF protein. It may be designed in any technique known to those skilled in the art, and its sequence, type of the nucleic acid (for example, it may include DNA or inosine), and modification are not particularly limited as long as it can function as an inhibitor. Further, it may inhibit the expression of the COUP-TF protein at either transcription level or translation level.

Examples of the inhibitor protein include a mutant COUP-TF protein that inhibits function of the COUP-TF wild-type protein competitively. For example, since the COUP-TF protein is a transcription regulator, forced expression of a recombinant mutant COUP-TF protein in cells, which has its DNA-binding domain and lacks its transcription inhibition domain, will inhibit binding of the endogenous COUP-TF protein to DNA competitively, resulting in inhibiting the function of the COUP-TF protein. For example, a DNA-binding domain of mouse COUP-TFI protein (NM_{—}010151) is the region at position 86 to 149 of the amino acid sequence of SEQ ID NO. 1, and that of COUP-TFII protein (NM_{—}009697) is the region at position 80 to 144 of the amino acid sequence of SEQ ID NO. 2; these regions may be used to construct a competitive inhibition mutant.

Further, as will be described in Examples, this competitive inhibition mutant may include a transcription activation domain. The transcription activation domain is not limited as long as it functions as an independent activation domain when bound with a DNA-binding domain of a heterologous protein, and any of known activation domains such as GAL4, Bicoid, c-Fos, B42 and VP16 may be used.

<Agent for Promoting Neuronal Differentiation and Method for Administering the Agent>

The inhibitor of the function of the COUP-TF protein may be formulated as a neuronal differentiation promoting agent using any of pharmaceutically acceptable carrier, diluent, or excipient known to those skilled in the art. This agent may be used as an experimental reagent in vitro or a medical drug in vivo.

When used as the experimental reagent, the agent inhibits neuronal differentiation of the NSPCs in various ways: if the inhibitor is a small molecule, it may be added to culture medium; if it is a nucleic acid, it may be used for transfection; and if it is a protein, an expression vector to express a gene encoding the protein can be transfected, or it can be made as a PTD fusion protein by using TAT domain etc., which is added to culture medium etc. In the case where the NSPCs are derived from ES cells or iPS cells, the inhibitor is preferably introduced into the cells at the stage of the primary neurosphere.

The disease to be targeted by the drug is not particularly limited as long as it is a defect requiring promotion of neuronal differentiation of the NSPCs for treatment in a human or non-human vertebrate, and examples of such defect include neurologic diseases and nerve injuries in central nervous system such as a cerebral infarction and a traumatic brain injury, brain ischemia, Huntington's disease and Alzheimer's disease as well as functional disorder by aging. The administration method and the dosage amount of the neuronal differentiation promoting agent for a patient can be appropriately chosen by those skilled in the art based on the administration purpose, the dosage form, the state of the patient etc.

It should be noted that the inhibitor may be used solely by itself or in a combination of multiple forms. For example, an inhibitor for COUP-TFI protein and an inhibitor for COUP-TFII protein may be used in a combination, or an inhibitor nucleic acid and an inhibitor protein for COUP-TF protein may be used in a combination.

Alternatively, an inhibitor which inhibits the functions of both the COUP-TFI protein and the COUP-TFII protein may be used. Such inhibitor may be designed by utilizing a conserved nucleotide sequence between the COUP-TF genes.

### EXAMPLE

### Example 1

### Knockdown of Coup-tf Gene in NSPCs

In this example, it is described that if a Coup-tf gene is knocked down in NSPCs, the cells preferentially differentiate into neurons when they are placed in a differentiation condition.

(1) Culture of Mouse ES Cells and Formation of Embryoid Bodies (EBs)

EB3 cells, which were obtained by inserting a Blasticidin-resistance gene at Oct3/4 locus of an ES cell line E14tg2a derived from a mouse strain 129/Ola to enable selection of undifferentiated ES cells, were cultured using Glasgow minimum essential medium (GMEM) supplemented with 10% FCS, non-essential amino acids, 1 mM sodium pyruvate, 0.1 mM 2-mercaptoehtanol and 1000 U/mL leukemia inhibitory factor (LIF) under the condition of 5% CO₂, 37° C.

Next, the ES cells were washed with PBS, dissociated by 0.25% trypsin-1 mM EDTA treatment, seeded in bacterial culture dishes at a concentration of 1×10⁵ cell/mL, and cultured in suspension for 4 to 8 days to allow formation of EBs using the culture supernatant as medium, which was obtained by transiently expressing the Xenopus Noggin gene in COS7 cells.

(2) Isolation of NSPCs from EBs by Selective Culture

The EBs thus formed were treated with 0.25% trypsin-1 mM EDTA solution and were dispersed into cells. The dispersed cells were seeded in bacterial culture dishes at a concentration of 5×10⁴ cells/mL in MHM medium, which is DMEM:F12(1:1) supplemented with glucose (0.6%), glutamate (2 mM), insulin (25 ug/mL), transferrin (100 ug/mL), progesterone (20 nM), putrescine (60 uM), selenium chloride (30 nM), FGF-2 (20 ng/mL) and heparin (2 ug/mL) and further added with mouse sonic hedgehog 1 protein (5 nM), and cultured in suspension for 7 to 9 days to allow formation of primary neurospheres.

(3) Knockdown of Coup-tf Gene

Sequences of shRNAs for knockdown of Coup-tfI gene and/or Coup-tfII gene were designed by using an online siRNA design program siDirect (http://design.mai.jp/). As for negative controls, pSilencer 2.1-U6 Negative Control (Ambion) as well as mutants of the shRNAs for Coup-tf knockdown, in which one, two or three bases were mutated were used. Their sequences are as follows.

KDI (for Coup-tfI):
(SEQ ID NO. 3)
GATGCTGCCCACATCGAAATTCAAGAGATTTCGATGTGGGCAGCATC
KDII (for Coup-tfII KD):
(SEQ ID NO. 4)
GTCCCAGTGTGCTTTGGAATTCAAGAGATTGGAAAGCACACTGGGAC
KDI + II (for Coup-tf KD):
(SEQ ID NO. 5)
GTCGAGCGGCAAGCACTACTTCAAGAGAGTAGTGCTTGCCGCTCGAC
2.1-U6:
(SEQ ID NO. 6)
ACTACCGTTGTTATAGGTGTTCAAGAGACACCTATAACAACGGTAGT

To express these shRNAs under the control of histone H1 promoter, their complementary sequences were synthesized and their double strand DNAs were formed by annealing, each of which was inserted between BglII site and XbaI site of an Entry Vector for shRNA pENTR4—H1. Each of the inserted shRNAs and histone H1 promoter were recombined into pCS-RfA-EG, a lentivirus vector for expressing shRNA, by using Gateway system (Invitrogen). The recombinant lentivirus vectors were introduced into 293T cells along with pCMV-VSV-G-RSV-rev and pCAG-HIVgp by using FuGENE6 (Roche), and the resulting culture supernatants were recovered, from which recombinant lentiviruses were purified at high concentration by ultracentrifugation. It should be noted that the lentivirus vectors contain the GFP gene to be expressed constitutively in order to enable to identify infected cells.

The recovered lentiviruses were infected to the neurospheres at MO125. Infected neurospheres were dispersed into single cells, and cultured in GMEM for 7 days to form secondary neurospheres. The neurospheres were subsequently passaged every 6 days.

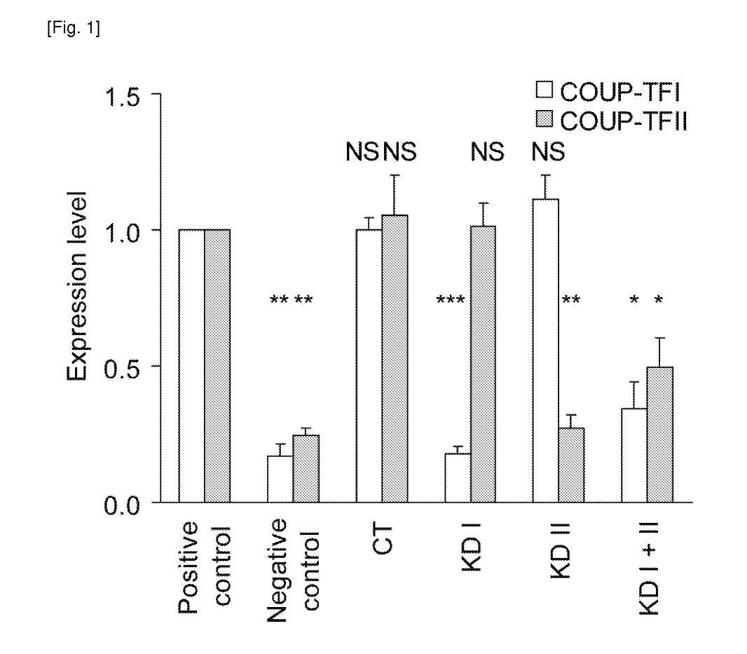

In order to confirm that the expression of the Coup-tf gene was inhibited by each of the shRNAs, the lentivirus vector expressing respective shRNA was introduced to Coup-tf expressing 293T cells to which the Coup-tf genes had been stably introduced to express both of the COUP-TF proteins and Western blotting was conducted on cell extracts of the shRNA-expressing cells. As for antibodies, anti-COUP-TFI antibody (mouse IgG, RRMX PP—H8132-00, 100-fold dilution) and anti-COUP-TFII antibody (mouse IgG, RRMX PP—H7147-00, 100-fold dilution) were used. Relative expression levels in respective cells of Negative control (wild-type 293T), CT (in which 2.1-U6 was introduced), KDI (in which KDI was introduced), KDII (in which KDII was introduced) and KDI+II (in which both KDI+KDII was introduced) were analyzed by t-test with normalizing the expression levels of each COUP-TF protein in Positive control (293T expressing Coup-tf) as 1.0, and plotted on a graph as shown in FIG. 1 (the result of p<0.05, p<0.01 and p<0.001 relative to Positive control are labeled by *, ** and *** respectively. Similar labeling by using * with regard to the p value was applied to the following graphs as well). As shown in these results, each shRNA of KDI, KDII and KDI+II was capable of inhibiting the expression of the COUP-TFI protein, the COUP-TFII protein and both of them, respectively.

(4) Analysis of Knocked-Down NSPCs

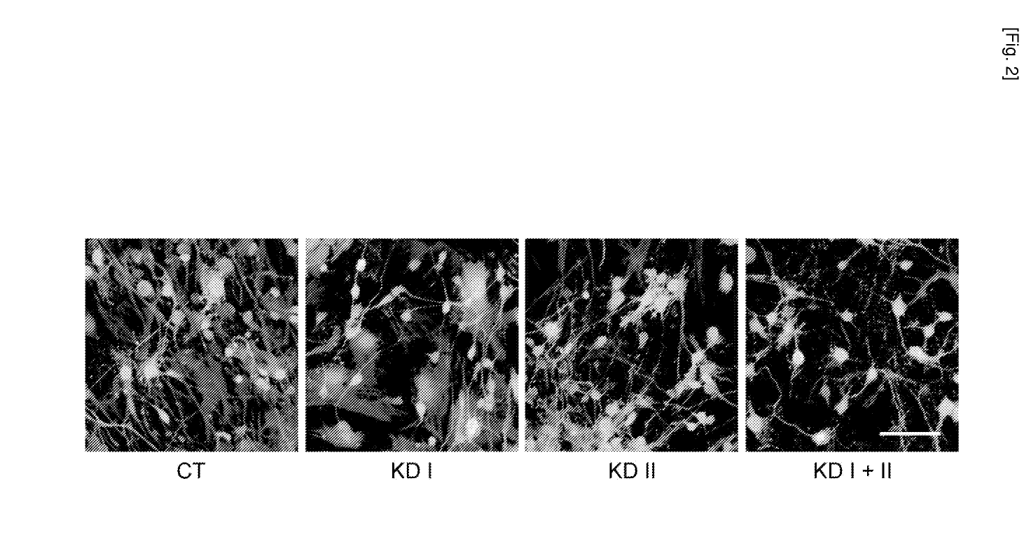

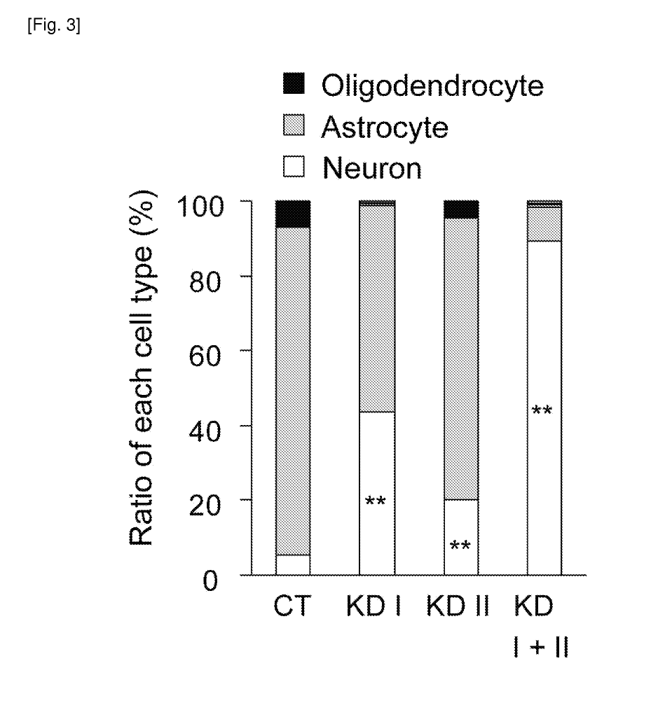

At each passage of neurospheres in which shRNAs had been introduced, a part of the neurospheres was dispersed by pipetting, seeded in a culture dish which was coated with poly-L-ornithin and filled with a differentiation medium, and grown in the presence of sonic hedgehog protein (5 nM) for 5 to 7 days to differentiate. Then, the cells were immunostained with anti-beta III tubulin antibody used as a marker for neurons (mouse IgG, Sigma T8660, 1000-fold dilution), anti-GFAP antibody as a marker for astrocytes (rabbit IgG, DAKO Z0334, 400-fold dilution) and anti-04 antibody as a marker for oligodendrocytes (mouse IgM, Chemicon MAB345, 8000-fold dilution) to observe their cellular morphology and the stained cell types by using fluorescent microscopy (FIG. 2). In each sample, cell numbers of neurons, astrocytes and oligodendrocytes were counted and ratios of each cell type were plotted on a graph (FIG. 3).

In each of the analyses for the cellular morphology, the staining patterns and the ratios of each cell type, it was shown that the ratio of neurons increased by inhibiting expression of the COUP-TFI protein or the COUP-TFII protein, and further increased by inhibiting both the COUP-TFI and COUP-TFII proteins. It should be noted that even when LIF and BMP2, factors which facilitate differentiation into glial cells, were added to the culture medium with introduction of the shRNAs, similar results were obtained. Further, the efficiency of the formation of neurospheres did not change by inhibiting expression of the COUP-TFI protein or the COUP-TFII protein.

Thus, by inhibiting function of at least one of the COUP-TF proteins in NSPCs in vitro, the cells have come to differentiate into neurons preferentially when placed in the differentiating condition.

Further, by utilizing the expression of Isl-1, a marker for early differentiating neurons in forebrain, hindbrain and spinal cord, it was confirmed that while neurospheres lose potential to differentiate into neurons which are differentiated during early developmental processes of the central nervous system every passage, neurospheres in which the function of the COUP-TF protein is inhibited maintain the potential to differentiate into such early differentiating neurons.

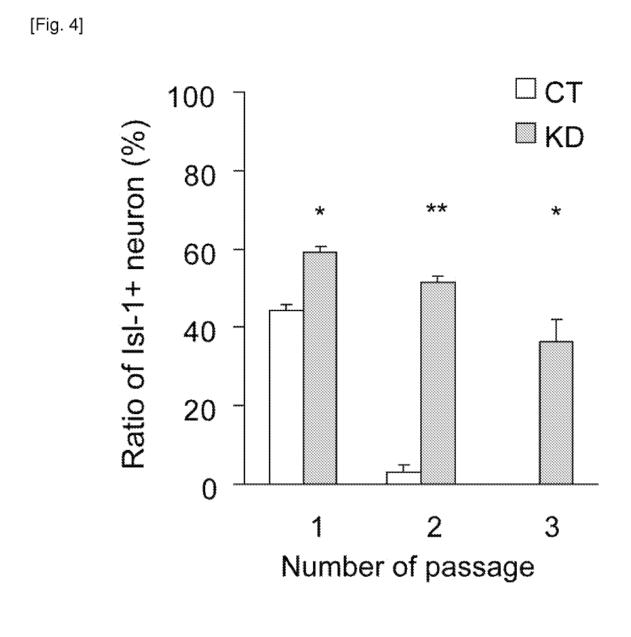

During passages of the neurospheres after the introduction of shRNAs, at each step of the first passage (to form the primary neurospheres), the second passage and the third passage, the cells were induced to differentiate and stained with anti-Is1-1 antibody (mouse IgG, DSHB 40.2D6, 200-fold dilution) to count the cell numbers of each cell type. Then the ratios of Isl-1 positive neurons against the number of total virus-infected neurons were calculated and plotted on a graph as shown in FIG. 4. In the untreated cells (CT), the expression of Isl-1 have been almost lost by the third passage, whereas in the cells to which shRNA KDI+II were introduced, the expression of Isl-1 was maintained at almost the same level even after the third passage.

Thus, the change of the neuronal type due to the induction of differentiation can be suppressed by the inhibition of the function of the COUP-TF protein.

### Example 2

### Knockdown of the Coup-tf Gene in Living Mice

In this Example, it is shown that during differentiation of NSPCs in a living mouse, knockdown of the Coup-tf gene induces neuronal differentiation.

(1) Mice and Viral Infection

In this Example, the shRNA-containing lentivirus as described in Example 1 was microinjected into cerebral ventricles of mouse embryos in uteri of ICR mice at 10.5 days or 12.5 days after fertilization. For the microinjection at 10.5 days, VS40 and Vevo660 (VisualSonics) was used.

(2) Analyses of Mouse Brain

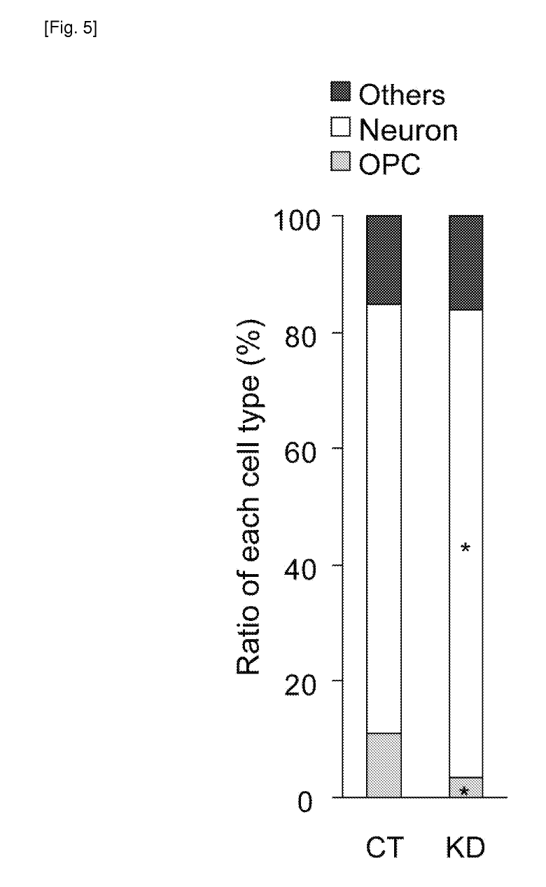

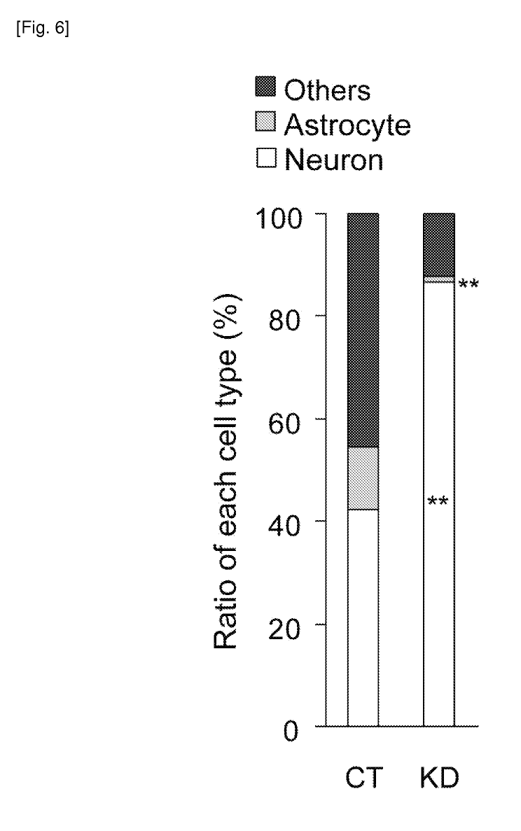

Immunohistochemical analyses were conducted at E16.5 for the brain of mice which had been infected with the shRNA-containing lentivirus at E10.5 (FIG. 5), as well as at P20 for the brain of mice which had been infected at E12.5 (FIG. 6). Cryosections of the brain were prepared and immunostaining was performed by standard protocols.

For the brain of 16.5-day embryo, anti-NeuN antibody was used as a marker for neurons; anti-Sox antibody and anti-Olig2 antibody were used as markers for oligodendrocyte precursor cells (OPCs), and double-positive cells were accounted as OPCs. Non-neurons other than OPCs were classified as “Others (other cells)”.

As shown in FIG. 5, the ratio of the OPCs in the cells to which shRNA KDI+II had been introduced (KD) decreased from 11% to 3.4% and the ratio of neurons increased from 73.8% to 80.4%, as compared with the negative control (CT) to which shRNA 2.1-U6 had been introduced.

For the mice at P20, the anti-NeuN antibody was used as a marker for neurons, the anti-GFAP antibody was used as a marker for astrocytes, and non-neurons which were negative for the anti-GFAP antibody were classified as “Others”.

As shown in FIG. 6, the ratio of astrocytes in the cells to which shRNA KDI+II had been introduced (KD) decreased from 12.2% to 1.3% and the ratio of neurons increased from 42.2% to 86.5%, as compared with the negative control (CT) to which shRNA 2.1-U6 had been introduced.

Thus, differentiation into neurons was promoted in vivo by inhibiting the function of the COUP-TF protein in the NSPCs.

(3) Analyses of Cell-Type of The Differentiated Neurons

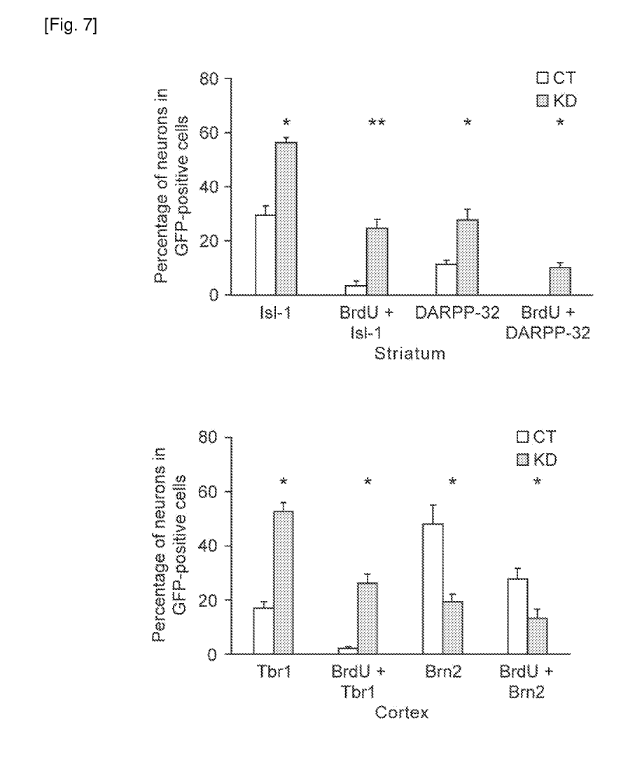

To determine the cell-types of the neurons that differentiated by inhibiting COUP-TF in vivo, immunohistochemical analyses were conducted at E16.5 for the brain of mice which had been infected with the shRNA-containing lentivirus at E10.5 (FIG. 7), using anti-Isl-1 antibody, anti-DARPP-32 antibody (Chemicon, 1/2000 dilution), anti-Tbr1 antibody (obtained from Dr. R. F. Hevner (Univ. of Washington, 1/10000 dilution) or anti-Brn2 antibody (Santa Cruz Biotechnology, 1/500 dilution). Cells born at E15.5 were labeled by BrdU incorporation and immunohistochemistry for BrdU was performed using anti-BrdU antibody (Abcam, 1/100 dilution) after pretreating the sections in 1N HCl at 37° C. for 30 min to denature the DNA. Negative control used was the same as used in (2).

As shown in FIG. 7, Isl-1 positive neurons and DARPP-32 positive neurons increased in striatum from 29.5% to 56.4% and from 11.5% to 27.7% as compared with the negative control (CT), respectively. The result of the double-staining with anti-BrdU antibody indicates that about half of the Isl-1 positive neurons and about one-third of the DARPP-32 positive neurons were born around E15.5. Tbr1 positive neurons also increased in cortex from 17.0% to 52.6% and about half of the Tbr1 positive neurons were born around E15.5 although they are normally barely generated after E15.5. Brn2 positive neurons, however, decreased from 48.1% to 19.2% in cortex, which might be sacrificed to the increase of Tbr1 positive neurons. It should be noted that the Isl-1 positive neurons in striatum are common precursors of cholinergic and GABAergic neurons and differentiated cholinergic neurons including motoneurons, the DARPP-32 positive neurons in striatum are GABAergic neurons, and the Tbr1 positive neurons in cortex are glutamatergic neurons. This differentiation pattern is consistent with the fact that neurospheres are induced to differentiate mainly into cholinergic neurons including motoneurons and GABAergic neurons in a general differentiation medium in vitro.

Thus the neurons that have differentiated by inhibiting the function of the COUP-TF protein in the NSPCs are at least partly Isl-1-positive, DARPP-32-positive or Tbr1-positive neurons, which should be cholinergic neurons, GABAergic neurons or glutamatergic neurons.

### Example 3

### Competitive Inhibition of DNA Binding Function of the COUP-TF Protein in NSPCs

In this Example, it is shown that neuronal differentiation can be promoted by competitive inhibition of the DNA binding function of the COUP-TF protein in NSPCs.

(1) Fusion Proteins

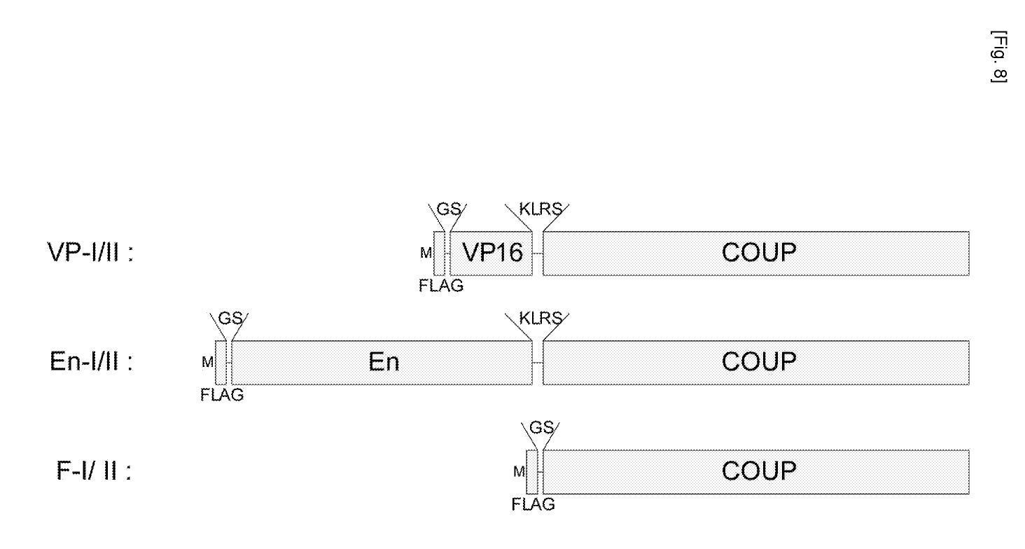

As for the fusion proteins to be used in this Example, either one of the DNA-binding domain at the N-terminal of the COUP-TFI protein (the amino acid sequence at position 2 to 403 of SEQ ID NO. 1) or the DNA-binding domain at the N-terminal of the COUP-TFII protein (the amino acid sequence at position 2 to 394 of SEQ ID NO. 2) was fused with either one of the transcription activation domain of VP16 (the amino acid sequence at position 413 to 490 of SEQ ID NO. 7 <P06492>) or the transcription repression domain of Drosophila Engrailed (En) protein (the amino acid sequence at position 2 to 295 of SEQ ID NO. 8 <NM 078976>) to construct either a dominant positive mutant (hereinafter referred to as VP-1 or VP-II) or a dominant negative mutant (hereinafter referred to as EnI or EnII) and used as respective mixtures (the mixed proteins are hereinafter referred to as VP-I/II and EnI/II, respectively). These fusion proteins do not have the transcription regulatory domain which is present at the N-terminus of the COUP-TF protein. As for control, COUP-TFI/II (a mixture of wild-type COUP-TFI and COUP-TFII proteins as well as F-1/II (a mixture of wild-type COUP-TFI and wild-type COUP-TFII, both of which are bound with a flag-tag (DYKDDDDK: SEQ ID NO. 9)) were used. Briefly, VP-1, VP-II, EnI and EnII have a structure represented as Met-FLAG-GlySer-VP/En-LysLeuArgSer-COUP, and F-I and F-II have a structure represented by Met-FLAG-GlySer-COUP (VP/EN indicates either one of VP-1, VP-II, EnI or EnII; and COUP indicates the DNA-binding domain of COUP-TFI or COUP-TFII). Schematic diagrams of these fusion proteins are shown in FIG. 8.

Recombinant DNAs encoding these fusion proteins were inserted instead of the shRNA in Example 1 into the lentivirus vector, and recombinant lentiviruses were produced by using 293T cells.

(2) Expression and Analyses of the Fusion Proteins in NSPCs

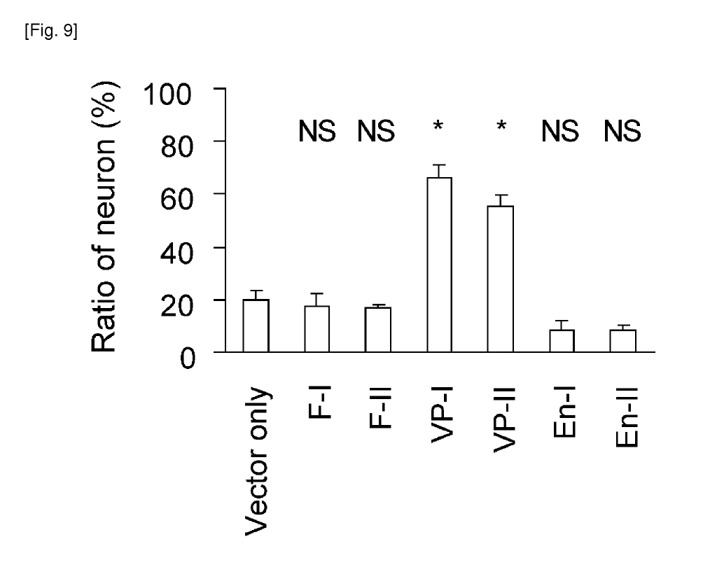

Like Example 1, by using the NSPCs derived from ES cells, primary neurospheres were infected with lentiviruses having the recombinant genes encoding the fusion proteins, and the cells were allowed to differentiate after the third passage, and ratios of neurons in the total cells were counted. The results were plotted on a graph as shown in FIG. 9.

Among the fusion proteins, only the dominant positive mutants (VP-1/II) promoted differentiation into neurons. This suggests that the COUP-TF protein functions as a repressor.

Thus, not only the inhibition of expression by using shRNAs but also the competitive inhibition against DNA binding by using the fusion protein can inhibit the function of the COUP-TF protein in the neural stem/progenitor cell, thereby promoting differentiation to neurons.

### INDUSTRIAL APPLICABILITY

The present invention enables to provide agents for promoting neuronal differentiation of NSPCs and methods therefor.

1. A method for promoting neuronal differentiation of a neural stem/progenitor cell comprising inhibiting function of a COUP-TFI protein and/or a COUP-TFII protein in the neural stem/progenitor cell.

2. The method according to claim 1, wherein expression(s) of (a) gene(s) encoding a COUP-TFI protein and/or a COUP-TFII protein are/is inhibited.

3. The method according to claim 1, wherein the function of a COUP-TFI protein and/or a COUP-TFII protein is inhibited by a nucleic acid capable of inhibiting expression of the gene.

4. The method according to claim 3, wherein the nucleic acid is a shRNA or a siRNA.

5. The method according to claim 1, wherein the function of a COUP-TFI protein and/or a COUP-TFII protein is inhibited by a competitive inhibition mutant of a COUP-TFI protein or a COUP-TFII protein.

6. The method according to claim 5, wherein the competitive inhibition mutant comprises a DNA-binding domain of a COUP-TFI protein or a COUP-TFII protein and lacks a transcription inhibition domain.

7. The method according to claim 1, wherein the neural stem/progenitor cell is derived from an embryonic stem cell or an induced pluripotent stem cell.

8. The method according to claim 7, wherein the neural stem/progenitor cell forms a primary neurosphere.

9. The method according to claim 1, wherein the differentiated neuron is Isl-1-positive, DARPP-32-positive or Tbr1-positive.

10. The method according to claim 1, wherein the differentiated neuron is a cholinergic neuron, a GABAergic neuron or a glutamatergic neuron.

11. A method for treating a patient with a disease for which promotion of neuronal differentiation of a neural stem/progenitor cell is required to be treated comprising inhibiting function of a COUP-TFI protein and/or a COUP-TFII protein in the neural stem/progenitor cell.

12. The method according to claim 11, wherein the disease is selected from the group consisting of brain ischemia, traumatic brain injury, Huntington's disease, and Alzheimer's disease.


SEQUENCE LISTING (9 sequences)

SEQ ID NO: 1 - protein, 420 aa, Mus musculus
MAMVVSSWRDPQDDVAGGNPGGPNPAAQAARGGGGGEQQQAGSGAPHTPQTPGQPGAPATPGTAGDKGQGPPGSGQSQQHIECVVCGDKSSGKHYGQFTCEGCKSFFKRSVRRNLTYTCRANRNCPIDQHHRNQCQYCRLKKCLKVGMRREAVQRGRMPPTQPNPGQYALTNGDPLNGHCYLSGYISLLLRAEPYPTSRYGSQCMQPNNIMGIENICELAARLLFSAVEWARNIPFFPDLQITDQVSLLRLTWSELFVLNAAQCSMPLHVAPLLAAAGLHASPMSADRVVAFMDHIRIFQEQVEKLKALHVDSAEYSCLKAIVLFTSDACGLSDAAHIESLQEKSQCALEEYVRSQYPNQPSRFGKLLLRLPSLRTVSSSVIEQLFFVRLVGKTPIETLIRDMLLSGSSFNWPYMSIQCS

SEQ ID NO: 2 - protein, 414 aa, Mus musculus
MAMVVSTWRDPQDEVPGSQGSQASQAPPVPGPPPGAPHTPQTPGQGGPASTPAQTAAGGQGGPGGPGSDKQQQQQHIECVVCGDKSSGKHYGQFTCEGCKSFFKRSVRRNLSYTCRANRNCPIDQHHRNQCQYCRLKKCLKVGMRREAVQRGRMPPTQPTHGQFALTNGDPLNCHSYLSGYISLLLRAEPYPTSRFGSQCMQPNNIMGIENICELAARMLFSAVEWARNIPFFPDLQITDQVALLRLTWSELFVLNAAQCSMPLHVAPLLAAAGLHASPMSADRVVAFMDHIRIFQEQVEKLKALHVDSAEYSCLKAIVLFTSDACGLSDVAHVESLQEKSQCALEEYVRSQYPNQPTRFGKLLLRLPSLRTVSSSVIEQLFFVRLVGKTPIETLIRDMLLSGSSFNWPYMAIQ

SEQ ID NO: 3 - DNA, 47 bp, Artificial Sequence
  primer
gatgctgcccacatcgaaattcaagagatttcgatgtgggcagcatc

SEQ ID NO: 4 - DNA, 47 bp, Artificial Sequence; identical to SEQ ID NO: 3
  primer

SEQ ID NO: 5 - DNA, 47 bp, Artificial Sequence
  primer
gtcgagcggcaagcactacttcaagagagtagtgcttgccgctcgac

SEQ ID NO: 6 - DNA, 47 bp, Artificial Sequence
  primer
actaccgttgttataggtgttcaagagacacctataacaacggtagt

SEQ ID NO: 7 - protein, 490 aa, herpes simplex virus 7
MDLLVDELFADMNADGASPPPPRPAGGPKNTPAAPPLYATGRLSQAQLMPSPPMPVPPAALFNRLLDDLGFSAGPALCTMLDTWNEDLFSALPTNADLYRECKFLSTLPSDVVEWGDAYVPERTQIDIRAHGDVAFPTLPATRDGLGLYYEALSRFFHAELRAREESYRTVLANFCSALYRYLRASVRQLHRQAHMRGRDRDLGEMLRATIADRYYRETARLARVLFLHLYLFLTREILWAAYAEQMMRPDLFDCLCCDLESWRQLAGLFQPFMFVNGALTVRGVPIEARRLRELNHIREHLNLPLVRSAATEEPGAPLTTPPTLHGNQARASGYFMVLIRAKLDSYSSFTTSPSEAVMREHAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 8 - protein, 552 aa, Drosophila melanogaster
MALEDRCSPQSAPSPITLQMQHLHHQQQQQQQQQQQMQHLHQLQQLQQLHQQQLAAGVFHHPAMAFDAAAAAAAAAAAAAAHAHAAALQQRLSGSGSPASCSTPASSTPLTIKEEESDSVIGDMSFHNQTHTTNEEEEAEEDDDIDVDVDDTSAGGRLPPPAHQQQSTAKPSLAFSISNILSDRFGDVQKPGKSMENQASIFRPFEASRSQTATPSAFTRVDLLEFSRQQQAAAAAATAAMMLERANFLNCFNPAAYPRIHEEIVQSRLRRSAANAVIPPPMSSKMSDANPEKSALGSLCKAVSQIGQPAAPTMTQPPLSSSASSLASPPPASNASTISSTSSVATSSSSSSSGCSSAASSLNSSPSSRLGASGSGVNASSPQPQPIPPPSAVSRDSGMESSDDTRSETGSTTTEGGKNEMWPAWVYCTRYSDRPSSGPRYRRPKQPKDKTNDEKRPRTAFSSEQLARLKREFNENRYLTERRRQQLSSELGLNEAQIKIWFQNKRAKIKKSTGSKNPLALQLMAQGLYNHTTVPLTKEEEELEMRMNGQIP

SEQ ID NO: 9 - protein, 8 aa, Artificial Sequence
  tag
DYKDDDDK